(19) United States
(12) Patent Application Publication
(10) Pub. No.: US 2025/0256757 A1
(43) Pub. Date: Aug. 14, 2025

(54) INTEGRATED STEERING WHEEL SENSORS FOR DRIVER SAFETY AND HEALTH MONITORING

(71) Applicant: Georgia Tech Research Corporation, Atlanta, GA (US)

(72) Inventors: Nasrin Hooshmand, Alpharetta, GA (US); Ashkan Zandi, Atlanta, GA (US)

(21) Appl. No.: 19/048,735

(22) Filed: Feb. 7, 2025

Related U.S. Application Data
(60) Provisional application No. 63/551,598, filed on Feb. 9, 2024.

(51) Int. Cl. B62D 1/04; A61B 5/00; A61B 5/33; G08B 21/04

(52) U.S. Cl. CPC B62D 1/046; A61B 5/33; A61B 5/4845; G08B 21/0461

(57) ABSTRACT

A steering wheel system is disclosed for monitoring a driver's vital signs of a driver. The steering wheel system comprises a steering wheel and sensors integrated into the steering wheel. The sensors are configured to detect one or more driver vital signs. The steering wheel system comprises a memory storing computer executable instructions, and a processor configured to execute the computer executable instructions. The processor receives the one or more vital signs from the array of sensors, wherein the one or more vital signs correspond to cortisol levels, alcohol levels, one or more proteins associated with an occurrence stroke, one or more biomarkers associated with myocardial infarctions, temperature, pulse rate, and oxygen levels. The processor determines a health risk of the driver based at least in part on the vital signs and alerts the driver in response to the health risk exceeding a certain health risk level.

### CROSS-REFERENCE TO RELATED APPLICATIONS

This application claims the benefit of priority to U.S. Provisional Patent Application No. 63/551,598 filed Feb. 9, 2024, the disclosure of which is incorporated herein by reference.

### FIELD OF INVENTION

The present disclosure generally relates to a steering wheel apparatus with a suit of sensors configured to measure one or more health parameters of a driver of a vehicle while the user is operating the vehicle.

### BACKGROUND

Modern day health-related sensors in motor vehicles measure the certain parameters associated with the driver's responsiveness, alertness, stress, fatigue, and/or inebriation. These sensing apparatuses are usually included in the steering wheel of a motor vehicle and measure the parameters based on a collection of sensing inputs positioned on the steering wheel. For example, U.S. Pat. No. 8,725,230 (Lisseman) refers to sensor assemblies mounted in a steering wheel. The assemblies have electrodes for measuring a biological parameter of a driver. The sensor assemblies are integrated into the outer rim of the wheel and are said to be capable of measuring parameters that give an indication of the driver's well-being, including heart rate, skin capacitance, skin temperature, and respiration rate. According to Lisseman, measured biological parameters of a driver, such as heart rate, skin capacitance, and/or skin temperature, can be used to determine if the driver is in a stressed state or is otherwise distressed.

U.S. Pat. No. 9,820,685 (Rake) refers to optical and electrical measurement devices for capturing parameters of a person in a motor vehicle. The devices are used to assess pulse rate/variability and arterial oxygenation saturation, and to collect electrocardiogram data. The devices include optical sensors which are attached to a steering wheel in transition zones between the wheel spokes and hub. According to Rake, bio-parameter monitoring can be used to care for patients with cardiac risk factors, to diagnose sleep disturbance, and to identify fatigue and stress.

U.S. Patent Application Publication No. 2023/0081472 (Wang) refers to an advanced driver assistance system (ADAS) configured to timely respond in case of an emergency. The system keeps track of the driver's health/consciousness by monitoring his or her vital signs (respiration rate, heart rate, and heart rate variability). According to Wang, continuous monitoring of the driver's status makes it possible for the ADAS to take control of the automobile in case of emergency, such as when the driver encounters a sudden heart attack, stroke, or fatigue, predicted or indicated by the driver's heart rate variability. According to Wang, heart rate variability, in combination with heart rate and respiration rate, can be an indicator of cardiac arrhythmia, alcohol usage, mental stress, drowsiness, and alertness.

### SUMMARY

The present disclosure relates, in general, to a steering wheel system with a number of health-related biosensors integrated therein. According to one aspect of the present disclosure, The steering wheel system can comprise an array of sensors integrated into the steering wheel, wherein each of the sensors are configured to detect one or more vital signs of the driver. The steering wheel system can further comprise a memory storing one or more computer executable instructions, and at least one processor configured to execute the one or more computer executable instructions. The at least one or more computer executable instructions can cause the at least one processor to receive the one or more vital signs from the array of sensors, wherein the one or more vital signs correspond to cortisol levels, alcohol levels, one or more proteins associated with an occurrence stroke, one or more biomarkers associated with myocardial infarctions, temperature, pulse rate, and oxygen levels. The at least one or more computer executable instructions can further cause the at least one processor to determine a health risk of the driver based at least in part on the vital signs, and alert the driver in response to the health risk exceeding a certain health risk level.

According to some aspects of the disclosure, the array of sensors can comprise one or more of a cortisol sweat sensor, alcohol sweat sensor, myocardial infarctions sensor, stroke sweat sensor, dehydration sweat sensor, pulse rate sensor, and oxygen level sensor. According to other aspects of the disclosure the array of sensors comprise an electrocardiogram sensor configured to monitor heart activity of the driver.

According to some aspects of the disclosure, the cortisol sweat sensor, alcohol sweat sensor, myocardial infarctions sensor, stroke sweat sensor, and dehydration sweat sensor comprise a flexible substrate, a plurality of nanostructures, a conductive electrode, counter electrode, work electrode, and reference electrode. The plurality of nanostructures can be configured to be slidable for replacement after usage.

According to some aspects of the disclosure, the at least one processor can determine that the alcohol levels are based at least in part on an interaction between ethanol molecules in sweat droplets of the driver and an electrode in an alcohol sweat sensor.

According to some aspects of the disclosure, the one or more proteins can be detected by a stroke sweat sensor based at least in part on an interaction between the one or more proteins in sweat droplets of the driver and an electrode in the stroke sweat sensor. The one or more proteins can comprise the S100B protein.

According to some aspects of the disclosure, the temperature and pulse rate of the driver can be determined using the same electrodes based on a position of at least one hand of the driver on the steering wheel.

According to some aspects of the disclosure, one or more of the array of sensors can be an electrochemical sensor. The electrochemical sensor can comprise a flexible substrate, one or more nanostructures, and one or more of a conductive electrode, counter electrode, working electrode, or a reference electrode.

According yet to another embodiment of the disclosure a vital sign monitoring sensor steering wheel is disclosed. The vital sign monitoring steering wheel can comprise a memory storing one or more computer executable instructions, and at least one processor configured to execute the one or more computer executable instructions. The at least one or more computer executable instructions can cause the at least one processor to receive one or more vital signs from at least one electrochemical sensor configured to detect one or more molecules in sweat from a hand of a driver on the vital sign monitoring sensor steering wheel. The at least one or more computer executable instructions can cause the at least one processor to determine a health risk of the driver based at least in part on the vital signs, and alert the driver in response to the determined health risk.

According to some aspects of this embodiment, the at least one electrochemical sensor can comprise a flexible substrate, one or more nanostructures, and one or more of a conductive electrode, counter electrode, working electrode, or a reference electrode. The working electrode can be coated with an enzyme that binds to molecules in a chemical present in the sweat of the driver. The molecules can be ethanol molecules. The working electrode can be coated with a binding molecule that binds to proteins present in the sweat of the driver. The proteins can be calcium-binding proteins. The proteins can be S100B proteins. The enzyme can be an alcohol dehydrogenase.

According to some aspects of this embodiment, the vital sign monitoring sensor steering wheel can comprise at least one electrocardiogram (ECG) sensor.

### BRIEF DESCRIPTION OF THE DRAWINGS

In order that the present disclosure may be readily understood, aspects of the steering wheel apparatus are illustrated by way of examples in the accompanying drawings, in which like parts are referred to with like reference numerals throughout.

FIG. 1 is drawing of an exemplary steering wheel illustrating a steering wheel integrated with a cortisol sweat sensor, an alcohol sweat sensor, an electrocardiogram (ECG) sensor, pulse rate sensor, temperature sensor, heart attack myocardial infarctions (MI) sweat sensor, stroke sweat sensor, and dehydration sensor in accordance with various aspects of the disclosure.

FIG. 2 is a drawing of an exemplary electrochemical sensor with slidably replaceable components in accordance with various aspects of the disclosure.

FIG. 3 is a drawing of an exemplary oxygen saturation level and pulse rate sensors integrated in a steering wheel in accordance with various aspects of the disclosure.

FIG. 4 is an exemplary schematic of electrochemical sensors for testing for chemicals in sweat in accordance with various aspects of the disclosure.

FIG. 5 is a drawing illustrating a steering wheel integrated with a cortisol sweat sensor, an alcohol sweat sensor, an electrocardiogram (ECG) sensor, pulse rate sensor, temperature sensor, heart attack myocardial infarctions (MI) sweat sensor, stroke sweat sensor, dehydration sensor, and oxygen level sensor, and optical pulse rate sensor in accordance with various aspects of the disclosure.

FIG. 6 is an exemplary flowchart of a process of issuing an in-vehicle alert to a driver in response to the driver's body signals being abnormal in accordance with various aspects of the disclosure.

FIG. 7 is an exemplary flowchart of a process of one or more sensors collecting and processing body signals from a driver in accordance with various aspects of the disclosure.

FIG. 8 is an exemplary simplified functional block diagram of an illustrative multifunctional electronic device in accordance with various aspects of the disclosure.

### DETAILED DESCRIPTION

The present disclosure also relates to an integrated steering wheel system configured with a paramount focus on driver safety and accident prevention. In the United States, road accidents have been a significant concern, leading to a substantial number of deaths and injuries annually. According to data from the National Highway Traffic Safety Administration (NHTSA) and the Centers for Disease Control and Prevention (CDC), there are tens of thousands of fatalities and millions of injuries resulting from accidents each year. On average, prior to 2022, there were over 30,000 motor vehicle-related fatalities reported annually in the U.S. Those accidents encompassed a wide range of factors, including but not limited to speeding, distracted driving, impaired driving, and lack of seatbelt use. Additionally, several million individuals sustained injuries in accidents, ranging from minor to severe, with many requiring medical attention and rehabilitation.

Those statistics underscore the importance of improving road safety and developing advanced driver assistance systems. Developing advanced driver assistance systems is essential. According to the present disclosure, a system incorporates a comprehensive array of sensors seamlessly embedded within the steering wheel, each strategically engineered to mitigate risks and enhance driver well-being, thereby safeguarding against sudden and unforeseen events. These integral sensors encompass: a cortisol sweat sensor, meticulously calibrated to assess stress levels by analyzing cortisol concentrations in the driver's sweat. Its role is pivotal in preempting stress-related distractions, reducing the likelihood of accidents. In some embodiments the cortisol sweat sensor can be based at least in part on an Epicore Biosystems (https://epicorebiosystems.com/) or GraphWear (https://graphwear.co/) sensor with a size of 1 centimeter by 1 centimeter.

An electrocardiogram (ECG) sensor continuously monitors the driver's ECG signals, affording crucial insights into heart health. In some embodiments, the ECG sensor can be based at least in part on analog devise Analog Devices AD8232 (Single-Lead ECG Front End) (https://www.analog.com/media/en/technical-documentation/data-sheets/AD8232.pdf). The size of the ECG sensor can be 4 millimeters by 4 millimeters in a LFCSP package. Rapid detection of cardiac anomalies significantly reduces the risk of sudden incapacitation while driving. A vital signs sensor, meticulously tracks vital parameters like body temperature, pulse rate, and oxygen saturation levels, serving as an early warning system for conditions that might lead to accidents. A myocardial infarctions (MI) sweat sensor is acutely tuned to detect specific biomarkers indicative of MI (heart attacks), thus preventing sudden health crises during driving. The approximate size of the MI sweat sensor can be 1 centimeter by 1 centimeter. In some embodiments, the stroke sweat sensor can be based at least in part on Laboratory ELISA kits, such as Abcam's S100B Human ELISA Kit (https://www.abcam.com/human-s100b-elisa-kit-ab234573.html). In other embodiments the stroke sweat sensor can be based at least in part on the principles discussed below about the stroke sweat sensor. The stroke sweat sensor can also be 1 centimeter by 1 centimeter.

In addition, there may be a stroke sweat sensor, with the ability to identify biomarkers associated with strokes and neurological events, preventing unforeseen incapacitation. An alcohol sweat sensor may proactively sense alcohol levels through sweat, enabling immediate assessment of driver impairment and reducing the likelihood of alcohol-related accidents. A dehydration sensor, based on sweat pH levels, provides crucial insights into the driver's hydration status, thus reducing the risk of health-related incidents caused by dehydration. The seamless integration of these sensors ensures real-time monitoring of the driver's physical and physiological conditions, offering a vital layer of protection against unforeseen health emergencies while behind the wheel. The system's ability to rapidly detect and respond to potential health hazards significantly contributes to accident prevention, thereby elevating driver safety to unprecedented levels. In some embodiments, the alcohol sweat sensor can be based at least in part on Winsen MQ-3 (breath-alcohol sensor) (http://www.winsen-sensor.com/sensors/alcohol-sensor/mq-3.html) and in some further embodiments the alcohol sweat sensor can be based at leas in part on BACtrack Skyn (transdermal wearable prototype) (https://www.bactrack.com/pages/bactrack-skyn). In some embodiments, the alcohol sweat sensor can be 1 centimeter by 1 centimeter, and yet still in other embodiments, the size of the alcohol sweat sensor is 5 millimeters by 5 millimeters. And the alcohol sweat sensor can be based on the principles discussed below.

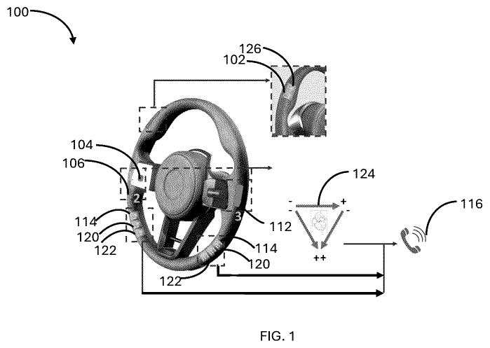

FIGS. 1, 3, and 5 are schematic illustrations of an example of a steering wheel constructed in accordance with the present disclosure. According to the present disclosure, the steering wheel may be provided with multiple health-related sensors, including one or more of a cortisol sweat sensor (for detecting driver stress), an alcohol sweat sensor, ECG (electrocardiogram) electrodes, a pulse rate sensor, a body temperature sensor, an MI (myocardial infarction) sweat sensor (for sensing conditions precedent to a heart attack), a stroke sweat sensor, a dehydration sweat sensor, and a blood-oxygen and pulse rate optical sensor.

In some embodiments, the temperature sensor can be based at least in part on a Texas Instrument (TI) sensor TI LMT70. The size of the temperature sensor can be at least 0.88 millimeters by 0.88 millimeters in a Die Size Ball Grid Array (DSBGA) package (https://www.ti.com/lit/ds/symlink/lmt70.pdf). The blood-oxygen and pulse rate optical sensor can be based at least in part on a Maxim Integrated MAX30102 sensor. The size of the blood-oxygen and pulse rate optical sensor can be 5.6 millimeters×3.3 millimeters×1.55 millimeters. These sensors require optical windows or direct skin contact on the steering wheel. They provide real-time measurement of pulse, oxygen saturation, and temperature with compact footprints.

In some embodiments, the MI sweat sensor be a troponin sensor that is based on Abbott i-TAT Troponin I test cartridges (blood-based, point-of-care) (https://www.pointofcare.abbott/us/en/offerings/istat-test-cards/istat-troponin-i). In other embodiments, the MI sweat sensor is based on the principles discussed below about the MI sweat sensor. The approximate size of the MI sweat sensor can be 1 centimeter by 1 centimeter.

In some embodiments, the dehydration sweat sensor can be based at least in part on IST AG LFA pH Series (ISFET-based) (https://www.ist-ag.com/en/lfa-ph-sensors). In other embodiments, the dehydration sweat sensor is based the principles discussed below about the dehydration sweat sensor. The size of the dehydration sweat sensor can be 1 millimeter by 1 millimeter.

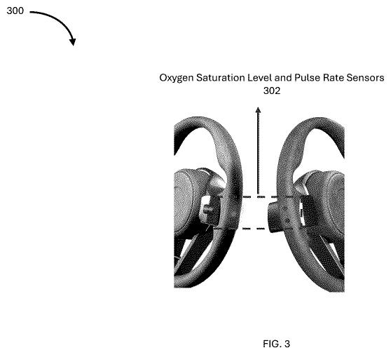

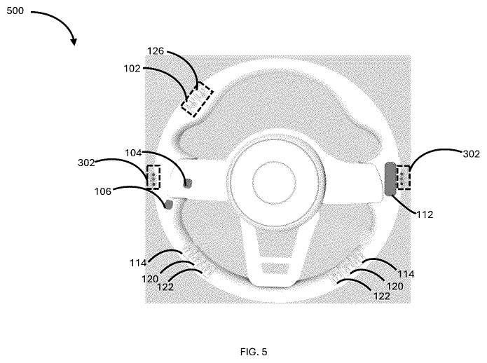

In the example discussed below, and as shown in FIG. 1 cortisol sweat sensor 102 and alcohol sweat sensor 126 are placed above and to the left of center of steering wheel 100, at about 10:30 if the steering wheel 100 were viewed as an analog clock face. That part of the steering wheel 100 is typically touched during engine startup. As a result, monitoring of driver stress and blood-alcohol concentration can occur early and proceed from that starting point. ECG electrodes 104 and 112 of ECG sensor 124, pulse rate sensor electrodes 106 and 112, and the oxygen saturation level and pulse rate optical sensors are positioned on both sides of the steering wheel. The oxygen saturation level and pulse rate optical sensors are not shown in FIG. 1, but are illustrated in FIG. 3 and FIG. 5 and described in the description of FIGS. 3 and 5. MI sweat sensor 114, stroke sweat sensor 120, and dehydration sweat sensor 122 are integrated into the steering wheel 100 beneath the other sensors. A temperature sensor can detect a temperature of the driver. The temperature sensor (not shown), can be co-located with the ECG electrodes 104 and 112 and the pulse rate sensor electrodes 106 and 112, or combined with the ECG electrodes 104 and 112 and the pulse rate sensor electrodes 106 and 112a. As soon as the driver's hand makes contact within any region of the ECG electrodes 104 and 112 or the pulse rate sensor electrodes 106 and 112 a local temperature sensor can read the driver's skin temperature from a palm or fingertips of the driver.

The ECG electrodes 104 and 112 can be placed so the driver's left hand or right hand makes contact (similar to ECG measurement). The pulse rate sensor electrode 106 can be used on the opposite side of the wheel for a second contact point.

Because the temperature sensors can be integrated into (or placed immediately beneath) these electrodes, any standard grip position covering electrodes 104, 106, or 112 will yield a temperature reading. If, for instance, the driver is only gripping electrodes 104 and 112, the integrated temperature sensors behind those electrodes will measure temperature. If their grip shifts to electrode 106, then the temperature sensor behind 106 will take over. This arrangement provides redundancy and ensures a reliable reading regardless of how the driver places their hands.

Temperature sensors can be miniaturized temperature sensors, integrated into specific contact points on steering wheel 100 where the driver's hands naturally rest. Temperature sensor [xxx] can continuously measure the driver's body temperature through his or her palms. This innovative approach enables temperature sensor [xxx] to capture subtle temperature variations, aiding in the early detection of fever or other temperature-related health issues.

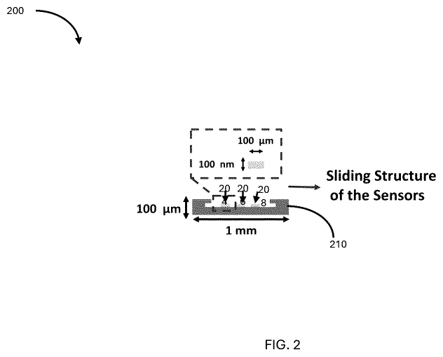

According to one aspect of the present disclosure, the organizational array of the sensors around the steering wheel promotes contact with the driver's hands at appropriate timing and facilitates maintenance of the sweat sensors which use consumable material and thus require replacement. In the illustrated example shown in FIG. 2, sensor 200 comprises electrodes 204, 206, and 208, which are positioned on substrate 210. Substrate 210 can be flexible and slidable, thereby enabling electrodes 204, 206, and 208 to be removed and replaced with another set of electrodes. In some embodiments, substrate 210 can be 1 millimeter in width and 1 micrometer in height. And each of electrodes 204, 206, and 208 can be 100 nanometers in height and 100 micrometers in width. In some embodiments, cortisol sweat sensor 102 and alcohol sweat sensors 126, MI sweat sensor 114, stroke sweat sensor 120, and dehydration sweat sensor 122 can be similar to design to that of sensor 200.

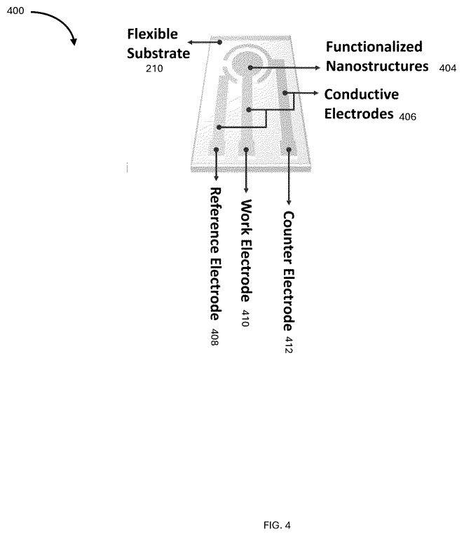

All three sensors, the MI sweat sensor 114, the stroke sweat sensor 120, and the dehydration sweat sensor 122, can share the same fundamental electrochemical sensor design shown, for example, in FIGS. 2 and 4. They each have a flexible substrate, work electrode, counter electrode, reference electrode, and can have a nanostructured or functionalized layer. The specific sensing chemistry (e.g., aptamers, enzymes, or other specialized coatings) that is tailored to detect a particular biomarker differentiates the MI sweat sensor 114, the stroke sweat sensor 120, and the dehydration sweat sensor 122.

The MI sweat sensor 114 targets cardiac troponins that appear in sweat during a heart attack (myocardial infarction). The electrode surfaces of the MI sweat sensor 114 are functionalized with aptamers or antibodies that selectively bind to troponins.

The stroke sweat sensor 120 can measure S100B protein, a stroke biomarker. The stroke sweat sensor 120 uses binding molecules (e.g., aptamers, antibodies) designed to specifically capture S100B.

The dehydration sweat sensor 122 can monitor pH changes (or related analytes) that signal dehydration. The dehydration sweat sensor 122 can use an ion-sensitive coating (e.g., ISFET or pH—responsive layer) to measure acid—base changes in sweat.

While the MI sweat sensor 114, the stroke sweat sensor 120, and the dehydration sweat sensor 122 may look the same externally, because each one is an electrochemical sensor with a similar electrode layout, they are formulated with different materials or binding chemistries for their unique health parameter.

FIG. 3 illustrates a steering wheel 300 including oxygen saturation level and pulse rate optical sensors 302 on either side of steering wheel 300. Oxygen saturation level and pulse rate optical sensors 302 can be located near ECG electrodes 104 and 112, thereby enabling the oxygen saturation level and pulse rate optical sensors and ECG electrodes 104 and 112 to share components and be operated simultaneously if desired. Oxygen saturation level and pulse rate optical sensors 302 can determine oxygen saturation levels in the driver's blood stream. In some embodiments, the driver can place his or her thumbs on designated spots on the steering wheel 300. These specific contact points incorporate conventional pulse oximetry technology, which uses light absorption to assess the oxygen saturation level in the driver's blood. This non-invasive method provides accurate and real-time oxygen level measurements, crucial for assessing respiratory health.

Oxygen saturation level and pulse rate sensors 302 can record the pulse rate through the same thumb contact points used for oxygen level measurement. That approach leverages photoplethysmography (PPG) technology, which involves shining light into the fingertip and measuring the variations in light absorption caused by blood flow. By analyzing those variations, oxygen saturation level and pulse rate sensors 302 can accurately record the driver's pulse rate, offering insights into heart rate and overall cardiovascular health. The collected data from the miniaturized temperature sensors, oxygen level measurement, and pulse rate recording is seamlessly integrated into the vehicle's central monitoring system. Real-time analysis of vital signs data allows for the immediate detection of anomalies or health concerns, ensuring the driver's safety and well-being throughout his or her journey. Any deviations from normal vital health parameters can trigger alerts or notifications, enabling timely medical attention when necessary, and contributing to the overall safety and health monitoring capabilities of the integrated steering wheel system.

Recording an ECG signal using a known commercial technology, similar to how it's done on devices like the Apple Watch, involves the integration of specialized electrodes and sensors to detect and measure the electrical activity of the heart. To adapt this technology for use in an integrated steering wheel system, the following mechanism can be applied. Within the steering wheel 100, are discreetly placed electrodes equipped with conductive materials which can serve as contact points for measurements of ECG sensor 124. When the electrodes come into contact with the driver's hands, primarily through the palms when gripping steering wheel 100, the driver's hands effectively complete a circuit of ECG sensor 124. Once the circuit of ECG sensor 124 is complete, electrical signals generated by the heart can be measured by ECG sensor 124. Steering wheel 100 can include integrated electronics including analog signal conditioning and amplification circuitry to process the detected electrical signals. A microcontroller or dedicated processing unit can further analyze and interpret the signals to generate an ECG waveform.

The measurements can then be transmitted to the vehicle's central monitoring system, or a connected device such mobile device 116, for real-time analysis and storage. In the event of irregularities or anomalies in the ECG, the vehicle's central monitoring system can trigger alerts or notifications to the driver, enhancing safety by allowing for immediate attention to potential cardiac issues. This adaptation of ECG recording technology into the steering wheel 100 ensures continuous, non-invasive monitoring of the driver's heart activity during the journey. It provides valuable insights into heart health and can serve as an additional layer of safety, helping to prevent accidents caused by sudden cardiac events. By detecting any high-risk abnormalities in the driver's ECG measurements, the processing unit can alert the patient's doctor, 911, or any emergency contacts of the driver, notifying them about the driver's condition, including the driver's exact location.

The vital signs sensor within the steering wheel 100 is designed to provide a comprehensive assessment of the driver's well-being while operating the vehicle. The vital signs sensor's functionality is initiated as soon as the driver places his or her hands on steering wheel 100, creating a seamless and non-invasive monitoring experience. The vital sign sensors measure key health indicators: specifically, body temperature, pulse rate, and oxygen saturation levels. The vital sign sensors can include the temperature sensor, the optical pulse-rate/oxygen-saturation sensor, and sometimes electrical pulse-rate sensors-all working in unison. For simplicity, the disclosure often collectively refers to this group of sensors as the vital sign sensor.

The MI sweat sensor 114 can be specifically designed to target cardiac troponins, which are biomarkers that are typically released into the bloodstream during a myocardial infarction (heart attack). However, it is noteworthy that cardiac troponins can also be present in sweat. That unique characteristic allows for the use of sweat analysis as an effective method for MI detection. By monitoring the concentration of cardiac troponins in sweat, the MI sensor can provide valuable insights into the driver's cardiac health, enabling the early detection of potential heart-related issues while they are operating a vehicle.

The core of MI sweat sensor 114 can be an electrochemical sensor that is meticulously calibrated to measure the concentration of cardiac troponins in sweat. That sensor consists of specialized electrodes, including a working electrode that is coated with troponin-specific binding molecules. When sweat comes into contact with the sensor's surface, any cardiac troponins present in the sweat will bind to those specific molecules on the working electrode. That binding event induces measurable electrochemical changes, such as changes in current or voltage. By quantifying those changes, the sensor can accurately determine the concentration of cardiac troponins in the sweat sample, providing a reliable means of MI detection in real-time.

In order to integrate MI sweat sensor 114 into the steering wheel 100, the following steps can be taken. A discreet and non-invasive sweat collection mechanism can be integrated into the steering wheel 100, preferably in areas where the driver's hands naturally make contact. Microfluidic channels or sweat collection points at those contact areas can be incorporated to efficiently direct sweat from the driver's palms to MI sensor 114. The MI sensor 114 can be securely embedded within the structure of the steering wheel 100 and can be within close proximity to the sweat collection points. The MI sensor 114 can be integrated with onboard electronics and data processing capabilities within the steering wheel 100. That allows for real-time analysis of electrochemical changes and the concentration of cardiac troponins in sweat. In some embodiments, a user-friendly interface is included within the vehicle's control system to display relevant information and alerts to the driver based on sensor readings generated by the MI sensor 114, such as notifying the driver of potential cardiac issues. The MI sensor 114 can be seamlessly incorporated into the steering wheel 100, offering continuous and non-invasive monitoring of cardiac health during every drive, and enhancing driver safety by enabling early MI detection.

Stroke sweat sensor 120 stands as an innovation in safeguarding driver well-being. Stroke sweat sensor 120 has been meticulously designed to detect the presence of S100B protein, a potential biomarker associated with stroke, in real-time during the act of driving. As the driver's hands make contact with the steering wheel 100, sweat from his or her palms is efficiently collected through non-invasive microfluidic channels. The collected sweat is then analyzed for elevated levels of S100B protein by specialized biomarker-binding molecules in the stroke sweat sensor 120. The S100B protein, a crucial biomarker for stroke diagnosis, holds a pivotal role in identifying and understanding this neurological condition.

S100B belongs to a family of calcium-binding proteins primarily found in the central nervous system, and its presence in bodily fluids, particularly blood and sweat, can provide valuable insights into brain-related events. In the context of stroke, S100B is released into the bloodstream when there is damage or injury to brain cells, often resulting from the interruption of blood flow to the brain, as seen in ischemic strokes or the rupture of blood vessels in hemorrhagic strokes. The detection of elevated levels of S100B in bodily fluids, such as sweat, serves as a critical indicator of potential brain damage or stroke occurrence.

This biomarker's unique ability to cross the blood-brain barrier and appear in sweat makes it a promising candidate for real-time stroke detection, particularly when integrated into innovative monitoring systems, like those within the steering wheel 100 of the vehicle, where swift detection of stroke-related biomarkers can help prevent life-threatening incidents while driving. Should stroke sweat sensor 120 detect a significant increase in S100B levels, it triggers immediate alerts or notifications, prompting the driver to take necessary action or pull over safely. This proactive approach not only enhances road safety but also underscores the potential of innovative biomarker-based monitoring to prevent strokes and related emergencies during the critical moments of driving.

The fabrication of cortisol sweat sensor 102 involves a precise and efficient process to ensure accurate detection of cortisol levels for enhanced driver well-being and safety. Cortisol sweat sensor 102 utilizes a specialized approach, harnessing the unique properties of cortisol aptamers for selective recognition and quantification of cortisol molecules. Those aptamers are carefully immobilized onto the surface of cortisol sweat sensor 102, providing high specificity for cortisol detection. The sweat collection mechanism of cortisol sweat sensor 102 is equally critical. To facilitate seamless and non-invasive sweat transfer, cortisol swat sensor 102 employs a microfluidic system integrated into steering wheel 100 or direct contact with the driver's sweat. In the former, the microfluidic channels discreetly channel sweat from the palm of the driver's hand to the active area of cortisol sweat sensor 102, thereby ensuring a continuous and controlled supply of sweat for analysis.

Alternatively, direct contact with the driver's sweat allows for immediate and localized sampling, enabling real-time monitoring without interruption. The combination of cortisol aptamers and precise sweat collection mechanisms ensures that the cortisol sweat sensor 102 can provide accurate and timely measurements of stress levels, contributing to the overarching goal of driver safety and accident prevention in steering 100. The proposed innovation of incorporating a thin layer of glucose with a gradually changing thickness onto the active part of cortisol sweat sensor 102, combined with the controlled dissolution of Nafion, presents an ingenious solution for extending the lifespan of cortisol sweat sensor 102 and ensuring long-term reliability. This mechanism not only allows for the preservation of sensor functionality but also ensures cost-effectiveness and sustainability. The gradual change in thickness of the glucose layer serves a dual purpose: initially protecting the active part of cortisol sweat sensor 102 from exposure to sweat, and later, as the Nafion dissolves upon sweat contact, enabling cortisol sweat sensor 102 to measure cortisol accurately.

This sequential process effectively extends the sensor's operational life by conserving the active area until it is needed for measurements. This sequential process refers to a layered approach for extending the life of the cortisol sweat sensor by applying a thin glucose layer and a partially dissolvable Nafion coating in series. The sequential process can include an initial protection comprising applying a thin layer of glucose that prevents the sensor's active area from being immediately exposed to sweat. As the Nafion dissolves upon exposure to sweat, the glucose layer gradually thins and is eventually removed. This can be referred to as controlled dissolution. The electrochemical sensor can be activated once the protective layers are sufficiently dissolved, the sensor's active surface is exposed, allowing cortisol measurements to begin only when needed. This staged (i.e., sequential) dissolution of glucose and Nafion extends the electrochemical sensor's operational lifespan because the sensing region is preserved until actual use, minimizing unnecessary wear or contamination.

By utilizing this innovative approach, the integrated steering wheel system can offer long-term health monitoring and stress detection capabilities without the need for frequent replacements or maintenance. This not only enhances driver safety but also underscores the practicality and efficiency of the system, making it a valuable addition to the automotive industry's pursuit of improved safety and well-being for drivers.

Detecting alcohol levels in sweat is a complex process that relies on the measurement of ethanol (alcohol) concentrations within the sweat samples. At the heart of the steering wheel 100 lies an advanced electrochemical alcohol sweat sensor (alcohol sweat sensor 126) which plays a pivotal role in enhancing driver safety and reducing the risk of alcohol-related accidents. The working electrode of alcohol sweat sensor 126 is meticulously functionalized with alcohol-specific recognition elements, typically enzymes like alcohol dehydrogenase. These recognition elements selectively interact with alcohol molecules present in the sweat, ensuring the specificity for ethanol in alcohol sweat sensor 126. As the driver grips the steering wheel 100, the alcohol sweat sensor 126 discreetly collects tiny sweat samples from their palms through microfluidic channels integrated into the contact points.

This non-invasive and seamless collection mechanism ensures that alcohol sweat sensor 126 can interact with the driver's sweat effectively. When ethanol molecules in the collected sweat samples come into contact with the recognition elements on the work electrode of alcohol sweat sensor 126, a highly specific electrochemical reaction is triggered. That reaction results in changes in electrical current or potential, directly proportional to the concentration of ethanol in the sweat. Alcohol sweat sensor 126 precisely measures the electrochemical response generated during the ethanol-specific reaction. That measured response serves as a direct indicator of the ethanol concentration present in the sweat sample.

The output of alcohol sweat sensor 126, contains information about the ethanol concentration, and is processed and analyzed in real-time by onboard electronics within steering wheel 100. Signal conditioning and data interpretation algorithms ensure accurate and rapid assessment of the driver's alcohol levels. If the measured ethanol concentration exceeds predefined safety thresholds, the system takes immediate action. It can generate a range of alerts or notifications, including visual and auditory cues to the driver, warning messages, or even more proactive measures such as preventing the vehicle from starting. This proactive approach ensures that drivers who may be impaired by alcohol are discouraged from operating a vehicle, thereby significantly reducing the likelihood of alcohol-related accidents.

Among the array of safety and health monitoring features incorporated into the steering wheel 100, an innovation comes in the form of a dehydration sensor 114. Dehydration sensor 114 relies on Ion-Sensitive Field Effect Transistor (ISFET) technology to measure and assess the driver's hydration status through sweat pH level analysis. By utilizing ISFETs, integrated within the microfluidic channels of the steering wheel 100, the dehydration sensor 114 can continuously and non-invasively monitor the pH levels of the driver's sweat. The ISFET-based pH measurement is exceptionally sensitive and precise, allowing the sensor to proactively detect any deviations associated with dehydration.

Ensuring optimal hydration is paramount for driver well-being, and dehydration sensor 114 conducts a real-time assessment which is a crucial tool in reducing the risk of health-related incidents attributed to dehydration. Whether issuing hydration reminders or taking immediate preventive measures, when necessary, this ISFET-enhanced dehydration sensor 114 exemplifies the steering wheel system's commitment to prioritizing driver health and safety on the road.

FIG. 4 is an exemplary schematic of an electrochemical sensor 400 that measures one or more chemicals in sweat of the driver in accordance with various aspects of the disclosure. The electrochemical sensor 400 can comprise a flexible substrate 210 upon which a reference electrode 408 rests, a work electrode 410 rests, a counter electrode 412 rests, a conductive electrode 406 rests, and functionalized nanostructures 404. The functionalized nanostructures 404 are nanoscale materials (e.g., nanoparticles, nanotubes, or porous films) that have been chemically functionalized, or coated, with a recognition element such as enzymes, aptamers, antibodies, or other binding molecules specific to a biomarker of interest. Their function is to capture or react with the target analyte (e.g., cardiac troponin, S100B protein, ethanol, cortisol) as sweat contacts the sensor surface. The high surface area and tailored chemical coatings greatly increase sensitivity and selectivity, allowing the sensor to detect very low concentrations of these biomarkers.

The conductive electrodes 406 are the electrical connections on the flexible substrate 210 linking the work electrode 404, the reference electrode 408, and the counter electrode 412 to circuitry outside the electrochemical sensor 400. For example, the conductive electrodes 406 can connect the other electrodes on the flexible substrate 210 to one or more amplifiers, microcontrollers, or other circuit elements or devices. The conductive electrodes 406 ensure reliable signal transmission between the sensor's active areas and the rest of the monitoring system.

The reference electrode 408 can set a stable baseline voltage, often via a silver/silver-chloride or similar electrode. It remains essentially unaffected by an analyte, letting the measuring electronics compare the working electrode's changes to a constant, known potential and thereby determine a concentration of a target molecule in the sweat of the driver.

The work electrode 410 is a primary sensing electrode, where the functionalized nanostructures 404 reside and where critical electrochemical reactions happen. Once target molecules bind to a functionalized coating of the functionalized nanostructures 404, the molecules cause a measurable change in current or potential on the work electrode 410. A signal associated with the current or potential of the work electrode 410 can be evaluated against a stable voltage of the reference electrode 408 to quantify an analyte concentration in real time.

The counter electrode 412, also known as the auxiliary electrode, completes a circuit for the electrochemical cell. The counter electrode 412 supplies or absorbs electrical current needed to balance the reaction at the work electrode 410 without affecting the reference potential.

Although each electrode has a distinct role, they operate together as a coherent electrochemical sensor, with the nanostructured coating of the work electrode 410 being the main location where the selective detection of the biomarker occurs. The reference electrode 408 provides a stable potential for comparison, and the counter electrode 412 balances the circuit current.

FIG. 5 is an exemplary illustrating a steering wheel 500 integrated with cortisol sweat sensor 102, alcohol sweat sensor 126, an electrocardiogram (ECG) sensor, pulse rate sensor, temperature sensor, heart attack myocardial infarctions (MI) sweat sensor 122, stroke sweat sensor 120, dehydration sensor 114, and oxygen saturation level and optical pulse rate sensor 302.

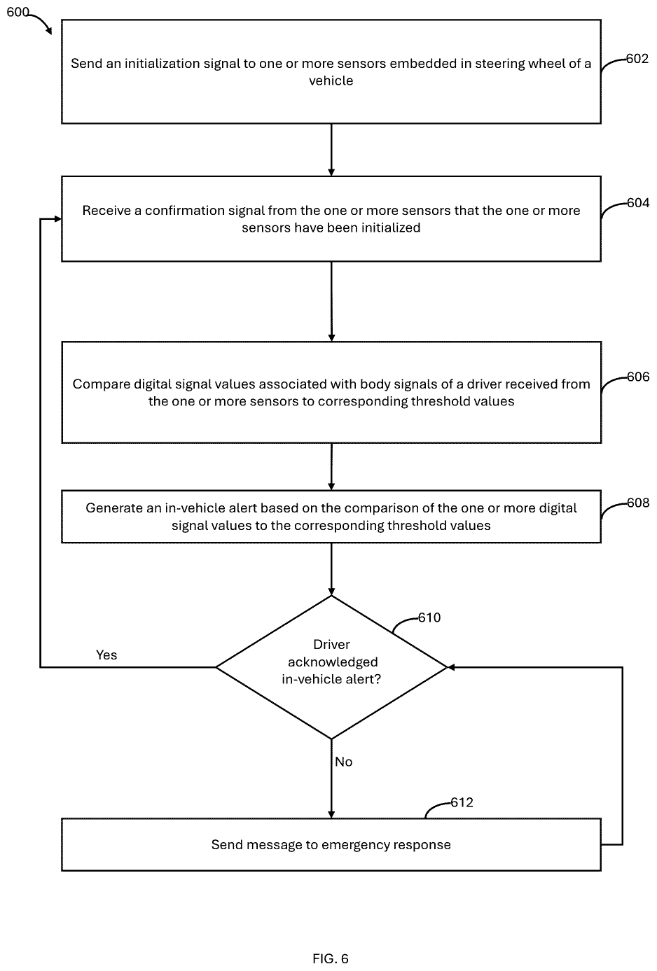

FIG. 6 illustrates a process 600 in which one or more vital signs associated with a driver are monitored and an in-vehicle alert or emergency response alert are generated when the one or more vital signs are not within a normal range of values.

At step 602 a processor, such as processor 710, in the multifunctional electronic device 700 can send an initialization signal to one or more sensors embedded in a steering wheel of a vehicle. Each of the one or more sensors (e.g., electrochemical sensors, ECG, or other vital signs) produces an electrical signal based on what it measures. The electrochemical sensors can measure alcohol, cortisol, MI troponin, stroke S100B, dehydration pH levels, and generate currents or voltages as a result of the chemical reaction happening at a work electrode.

An ECG sensor can produce a low-level voltage waveform corresponding to the heart's electrical activity. A vital sign sensor, such as a pulse oximetry/optical sensor, can measure light intensity changes (photodiode current) associated with blood volume changes. Another vital sign sensor, such as a temperature sensor, can measure small voltages or changes in resistance from a temperature-sensitive element, such as like a thermistor or dedicated integrated circuit (IC). The initialization of the one or more sensors can include powering on the one or more sensors, and setting up communication protocols (SPI, I²C, CAN, etc.) with each sensor.

At step 604 the processor can receive a confirmation signal from the one or more sensors that the one or more sensors have been initialized. The processor can execute one or more algorithms in order to process each of the different sensor's data. The processor can process electrochemical sensor signals by interpreting current or voltage vs. time to determine analyte concentration. For example, if current from the electrochemical sensor that measures alcohol content is above a threshold, that can indicate a high concentration of ethanol in the sweat of the driver. The processor can process ECG waveforms to detect R-peaks, measure intervals (e.g., QRS duration), and compute heart rate or detect arrhythmias. The processor can process vital sign sensor signals such as pulse oximetry (optical) signals, temperature signals, and electrical pulse rate sensor signals. The optical pulse oximetry signals can be calculations based on absorbed/reflected light to find SpO₂ and pulse rate. The temperature signals can be measured by a thermistor or solid state sensor and the measured signals can be compared against normal ranges of values. The processor can process electrical pulse rate sensor signals based on or derived from amplitude or timing of detected electrical pulses generated by the electrical pulse rate sensor.

At step 606 the processor can compare digital signal values associated with body signals of a driver that are received from the one or more sensors to threshold values. The processor can compare results (e.g., troponin level, spO₂, ECG anomalies) to predefined thresholds. If any of the compared results exceeds a critical threshold (e.g., dangerously high troponin or abnormal ECG), the processor can generate an in-vehicle alert-which may be visual, auditory, or integrative (e.g., locking the ignition, calling emergency contacts, notifying a telemedicine service, etc.) at step 608.

At step 610 the processor can determine whether the driver has acknowledged the in-vehicle alert. If the processor receives a signal from an input from the driver indicating that the driver has acknowledged the in-vehicle alert the processor can return to step 604. The input can be a button the steering wheel or on a console of the vehicle. In some embodiments, the input can be a change in compared results determined in step 608 in which the compared results no longer exceed the critical threshold. If, however, the driver does not acknowledge the in-vehicle alert the processor can send a message to an emergency response system such as 911 at step 612. After the processor sends the message, the processor can return to step 610 until the driver acknowledges the in-vehicle alert or the compared results no longer exceed the critical threshold.

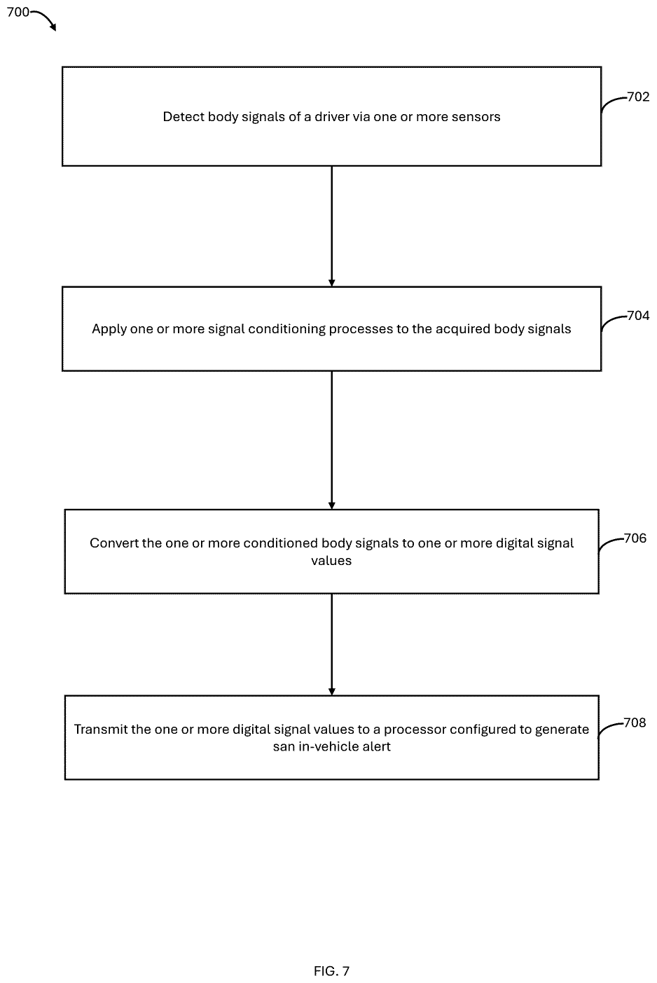

FIG. 7 is an exemplary flowchart of a process of one or more sensors collecting and processing body signals from a driver in accordance with various aspects of the disclosure. At step 702 the one or more sensors can detect body signals of a driver. The one or more body signals can include ECG signals, vital sign signals, or electrochemical signals.

At step 704 the one or more sensors processor can condition or filter the detected body signals. Because the detected body signals are raw signals, they can be small in magnitude, or can include noise (e.g., from movement or external interference). In response an analog front end of each of the one or more sensors can include an amplifier that amplifies the detected body signals into a measurable voltage/current range. The detected body signals can also be filtered to remove unwanted noise (e.g., high-frequency interference, baseline drift). The detected body signals can also be offset adjusted in order to shift a baseline of the detected body signals to a suitable operating range for digitization.

At step 706 the detected body signals can be converted to digital signals using a analog-to-digital converter (ADC) in the front end of each of the one or more sensors. In some embodiments, the conditioned, filtered, and/or offset adjusted detected body signals can be digitized by a processor that is not the same as the processor in each the one or more sensors. For example, the processor that generates an in-vehicle alert in accordance with process 600 can receive the conditioned, filtered, and/or offset adjusted detected body signals and an ADC associated with the processor that generates the in-vehicle alert can convert the analog signals into digital values.

At step 708, the one or more digital signal values can be transmitted over an internal bus (e.g., SPI, I²C) or direct ADC lines to the processor that generates the in-vehicle alert.

The one or more digital signal values can be stored locally (e.g., in on-board memory) of the one or more sensors, and/or transmitted sent to an external system (e.g., to a mobile app, cloud server, or directly to storage maintained by an emergency response service such as 911 in cases of emergency).

It should be noted that there are some differences in the way electrochemical, ECG, and vital sign signals are processed. Electrochemical sensors generate signals based on a redox current or voltage. A front end of the electrochemical sensor can include an electrochemical interface circuit (e.g., potentiostat) that measures and controls the voltage between the work and reference electrodes, then tracks current flowing through the counter electrode. If the electrochemical sensor includes an ADC, the analog signals corresponding to the measurements of the current are digitized by the ADC. and analyzed to quantify the concentration of biomarkers (e.g., troponin, cortisol, or ethanol). In some embodiments, the magnitude of the current or potential is monitored for changes over time.

ECG sensors generate a continuous low-voltage waveform (millivolt range). A front-end of the ECG sensors can include a high-input-impedance instrumentation amplifier with band-pass filters to eliminate baseline drift and high-frequency noise (e.g., 50/60 Hz interference). The ECG sensors include microcontrollers that extract heart rate, detects arrhythmias, and identifies wave patterns (e.g., P waves, QRS waves, and T waves).

Vital signs sensors can include optical pulse rate/SpO₂ (Pulse Oximeter) sensors, which use an LED+photodiode arrangement. The raw output of vital signs sensors can be a varying current in response to changes in blood absorption. A dedicated optical analog front end can modulate the LED and samples the photodiode output. Vital signs sensors can also include temperature which can be obtained from a thermistor or solid-state sensor. The thermistor or solid-state sensor can provide an analog signal or direct digital output.

Vital signs sensors can also include an electrical pulse rate sensor, which is similar to an ECG sensor but simpler in scope. The electrical pulse rate sensor can rely on measuring fluctuations in skin impedance or a partial ECG lead to detect heartbeats.

Despite these differences, once signals are conditioned, filtered, and converted to digital form, the central monitoring system's overarching flow—collect, compute, compare to thresholds, and alert if necessary—remains consistent.

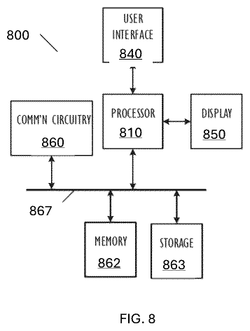

Referring now to FIG. 8, a simplified functional block diagram of an illustrative multifunctional electronic device 800, according to various aspects of the disclosure, is shown. Multifunction electronic device 800 may include processor 810, memory 820, storage device 830, user interface 840, display 850, communications circuitry 860, and communications bus 850. Multifunction electronic device 800 may be, for example, a personal electronic device such as a Personal Digital Assistant (PDA), mobile telephone, or a tablet computer.

Processor 810 may execute instructions necessary to carry out or control the operation of many functions performed by device 800. Processor 810 may, for instance, drive display 850 and receive user input from user interface 840. User interface 840 may allow a user to interact with device 800. For example, user interface 840 can take a variety of forms, such as a button, keypad, dial, a click wheel, keyboard, display screen and/or a touch screen. Processor 810 may also, for example, be a system-on-chip such as those found in mobile devices and include a dedicated Graphics Processing Unit (GPU). Processor 810 may be based on Reduced Instruction-Set Computer (RISC) or Complex Instruction-Set Computer (CISC) architectures or any other suitable architecture and may include one or more processing cores.

Memory 820 may include one or more different types of media used by processor 810 to perform device functions. For example, memory 820 may include memory cache, Read-Only Memory (ROM), and/or Random-Access Memory (RAM). Storage 830 may store media (e.g., audio, image and video files), computer program instructions or software, preference information, device profile information, and any other suitable data. Storage 830 may include one more non-transitory storage mediums including, for example, magnetic disks (fixed, floppy, and removable) and tape, optical media such as Compact Disc-Read Only Memorys (CD-ROMs) and Digital Video Disks (DVDs), and semiconductor memory devices such as Electrically Programmable Read-Only Memory (EPROM), and Electrically Erasable Programmable Read-Only Memory (EEPROM). Memory 820 and storage 830 may be used to tangibly retain computer program instructions or code organized into one or more modules and written in any desired computer programming language. When executed by, for example, processor 810 such computer program code may implement one or more of the methods described herein.

The present disclosure represents a novel advancement in automotive safety technology, promising not only safer roads but also greater peace of mind for drivers, as it significantly reduces the risks associated with sudden and unexpected events while driving.

The integrated steering wheel sensor technology described by way of example herein presents a wide array of cofercial and other applications that span various sectors, enhancing overall safety and efficiency. In the automotive industry, the technology can be integrated into new cars, significantly enhancing driver safety by monitoring health and physiological conditions in real-time. Its potential extends to health monitoring, especially beneficial for professional drivers and individuals with pre-existing health conditions, ensuring their well-being while on the road.

For the insurance and fleet management sectors, systems constructed in accordance with the present disclosure may offer substantial benefits. By ensuring driver health and safety, they can lead to reduced insurance premiums and more effective fleet management, as they minimize the risks associated with health-related driving impairments. Additionally, such systems may play a crucial role in road safety initiatives. By proactively preventing accidents caused by health-related issues or impairment, they may contribute significantly to the broader goal of enhancing road safety for all users.

Systems and methods in accordance with the present disclosure have many advantages over known systems and methods, including, for example: (1) Continuous monitoring offers real-time, non-invasive monitoring of various health parameters while driving; and (2) multi-modal sensing, integration, and non-invasiveness may be highly advantageous. Sensors for such systems and methods may be integrated into the steering wheel, making them more practical and user-friendly than separate monitoring devices. Also, they may provide a unique ability to monitor a wide range of health parameters, unlike existing technologies that focus on a single aspect.

Systems and methods in accordance with the present disclosure may be constructed and operated to achieve a number of goals, including the following: (1) Accuracy and Reliability-Ensuring sensors provide accurate readings across diverse conditions and individuals; (2) Privacy and Security-Safeguarding sensitive driver data against breaches; (3) Maintenance and Durability-Ensuring long-term functionality and resilience of embedded sensors; and (4) User Interface Integration: Providing an intuitive interface without distracting drivers.

1. A steering wheel system for monitoring vital signs of a driver, the steering wheel system comprising:
a steering wheel;
an array of sensors integrated into the steering wheel, wherein each of the sensors are configured to detect one or more vital signs of the driver;
a memory storing one or more computer executable instructions; and
at least one processor configured to execute the one or more computer executable instructions thereby causing the at least one processor to:
receive the one or more vital signs from the array of sensors, wherein the one or more vital signs correspond to cortisol levels, alcohol levels, one or more proteins associated with an occurrence stroke, one or more biomarkers associated with myocardial infarctions, temperature, pulse rate, and oxygen levels;
determine a health risk of the driver based at least in part on the vital signs; and
alert the driver in response to the health risk exceeding a certain health risk level.

2. The steering wheel system of claim 1, wherein the array of sensors comprise one or more of a cortisol sweat sensor, alcohol sweat sensor, myocardial infarctions sensor, stroke sweat sensor, dehydration sweat sensor, pulse rate sensor, and oxygen level sensor.

3. The steering wheel system of claim 1, wherein the array of sensors comprise an electrocardiogram sensor configured to monitor heart activity of the driver.

4. The steering wheel system of claim 2, wherein the cortisol sweat sensor, alcohol sweat sensor, myocardial infarctions sensor, stroke sweat sensor, and dehydration sweat sensor comprise a flexible substrate, a plurality of nanostructures, a conductive electrode, counter electrode, work electrode, and reference electrode.

5. The steering wheel system of claim 4, wherein the plurality of nanostructures are configured to be slidable for replacement after usage.

6. The steering wheel system of claim 1, wherein the alcohol levels are determined based at least in part on an interaction between ethanol molecules in sweat droplets of the driver and an electrode in an alcohol sweat sensor.

7. The steering wheel system of claim 1, wherein the one or more proteins are detected by a stroke sweat sensor based at least in part on an interaction between the one or more proteins in sweat droplets of the driver and an electrode in the stroke sweat sensor.

8. The steering wheel system of claim 7, wherein the one or more proteins comprise the S100B protein.

9. The steering wheel system of claim 1, wherein the temperature and pulse rate of the driver can be determined using the same electrodes based on a position of at least one hand of the driver on the steering wheel.

10. The steering wheel system of claim 1, one or more of the array of sensors is an electrochemical sensor.

11. The steering wheel system of claim 10, wherein the electrochemical sensor comprises a flexible substrate, one or more nanostructures, and one or more of a conductive electrode, counter electrode, working electrode, or a reference electrode.

12. A vital sign monitoring sensor steering wheel, the vital sign monitoring steering wheel comprising;
a memory storing one or more computer executable instructions;
at least one processor configured to execute the one or more computer executable instructions thereby causing the at least one processor to:
receive one or more vital signs from at least one electrochemical sensor configured to detect one or more molecules in sweat from a hand of a driver on the vital sign monitoring sensor steering wheel;
determine a health risk of the driver based at least in part on the vital signs; and
alert the driver in response to the determined health risk.

13. The vital sign monitoring sensor steering wheel of claim 12, wherein the at least one electrochemical sensor comprises a flexible substrate, one or more nanostructures, and one or more of a conductive electrode, counter electrode, working electrode, or a reference electrode.

14. The vital sign monitoring sensor steering wheel of claim 13, the working electrode is coated with an enzyme that binds to molecules in a chemical present in the sweat of the driver.

15. The vital sign monitoring sensor steering wheel of claim 14, wherein the molecules are ethanol molecules.

16. The vital sign monitoring sensor steering wheel of claim 13, the working electrode is coated with a binding molecule that binds to proteins present in the sweat of the driver.

17. The vital sign monitoring sensor steering wheel of claim 16, wherein the proteins are calcium-binding proteins.

18. The vital sign monitoring sensor steering wheel of claim 17, wherein the proteins are S100B proteins.

19. The vital sign monitoring sensor steering wheel of claim 14, enzyme is a alcohol dehydrogenase.

20. The vital sign monitoring sensor steering wheel of claim 12, further comprising at least one electrocardiogram (ECG) sensor.
